(19) United States
(12) Patent Application Publication
(10) Pub. No.: US 2006/0035316 A1
(43) Pub. Date: Feb. 16, 2006

(54) Plasmids expressing human insulin and the preparation method for human insuling thereby

(76) Inventors: Sang-Yong Lee, Seoul (KR); Sung-Jin Oh, Seoul (KR); Chang-Kyu Kim, Kyunggi-do (KR); Young-Jin Son, Kyunggi-do (KR); Kyong-Hee Park, Kyunggi-do (KR); Cheol-Ki Min, Seoul (KR); Byung-Min Chol, Seoul (KR); Tae-Won Kang, Seoul (KR); Jung-Woo Kim, Seoul (KR)

Correspondence Address: BIRCH STEWART KOLASCH & BIRCH, PO BOX 747, FALLS CHURCH, VA, 22040-0747, US

(21) Appl. No.: 10/487,746

(22) PCT Filed: Nov. 12, 2003
(86) PCT No.: PCT/KR03/02427

§ 371 Date: Apr. 2, 2004

(30) Foreign Application Priority Data
Nov. 12, 2002 (KR) 10-2002-0070188
Nov. 11, 2003 (KR) 10-2003-0079366

(51) Int. Cl. C07H 21/04; C12P 21/06; C07K 14/62

(52) U.S. Cl. 435/69.1; 435/320.1; 435/325; 530/303; 536/23.5

(57) ABSTRACT

The present invention relates to human insulin expression plasmids and a method for producing insulin using the same. The plasmids comprise a sequence encoding a compound of the formula R—B—X-A, in which R is a leader peptide of the formula of Met-Thr-Met-Ile-Thr-Y (SEQ ID NO: 36), in which Y is one selected from lysine, arginine, a peptide containing lysine as an amino acid at its C-terminal, or a peptide containing arginine as an amino acid at its C-terminal; B is human insulin B-chain or analogue thereof; X is a peptide connecting B with A; and A is human insulin A-chain or analogue thereof. The method for preparing insulin using the plasmids according to the present invention converts the proinsulin fusion protein into human insulin in a single enzymatic cleavage process and minimizes the generation of by-products after the enzymatic cleavage, thereby producing insulin at a high yield. Therefore, the plasmids according to the present invention and the method for preparing insulin using the same can be usefully applied to the industrial mass-production of human insulin.

### BACKGROUND OF THE INVENTION

1. Field of the Invention

The present invention relates to plasmids for expression of human insulin and a method for preparing insulin using the same.

2. Description of the Related Art

Insulin is a hormone secreted in the pancreas to regulate the glucose level in blood and binds to insulin receptors on the cell surfaces, thereby promoting the use of glucose and reducing the blood glucose level. Now, it is widely used as a therapeutic agent of diabetes. Insulin is produced as a precursor form in the pancreas. Proinsulin comprises an A-chain, a B-chain, and a C-chain connecting the two chains. When the C-chain is cut off in the cell, proinsulin is converted into active insulin comprising only the A-chain and the B-chain.

As the genetic engineering technology develops, various recombinant proteins can be mass-produced using E. coli transformed with recombinant plasmids. One of the most important problems in the production of the recombinant proteins is that the proteins have short half life in the host cells (Talmadge K, et al. Proc Natl Acad Sci USA. 1982;79:1830-3, Shen S H. Proc Natl Acad Sci USA. 1984;81:4627-31). For example, the half life of rat proinsulin in E. coli has been reported to be about 2 minutes (Talmadge K, et al. Proc Natl Acad Sci USA. 1982;79:1830-3).

The degradation of expressed proteins is closely related to the folding of the proteins. Cells degrade proteins with an incomplete tertiary structure or damaged, and convert them into amino acids, whereby the intracellular composition can be efficiently used. In the cytoplasm of E. coli, the initial protein degradation is performed by HSPs (heat shock proteins) using ATP.

A method that includes expressing a protein in the form of inclusion body, followed by refolding it to recover its activity may be used to increase the stability of the recombinant protein. Generally, the inclusion body is not affected by proteases and can be accumulated to a high concentration up to 50% of intracellular proteins. Accordingly, the expression of a target protein in the form of inclusion body would be a very excellent method which can economically produce the target protein, if an efficient refolding process for the formation of the correct tertiary structure of the protein is developed (Mukhopadhyay A. Adv Biochem Eng Biotechnol. 1997;56:61-109).

In the production of human insulin in E. coli, the above-described method has been broadly applied. The commonly used methods are expressing recombinant insulin in the form of a fusion protein to increase stability, followed by chemical cleavage. For example, proinsulin gene is inserted into a plasmid, containing a gene of a protein having a high stability in E. coli such as β-galactosidase, to construct a recombinant plasmid and the proinsulin fusion protein is expressed in E. coli transformed with the plasmid.

According to the above-described method, in order to prepare human insulin by purification of the proinsulin fusion protein, the bodies are purified to increase the purity of the target protein and the washed inclusion bodies are dissolved by a treatment with a denaturant and subjected to sulfonation to minimize the formation of hydrophobic interaction and the wrong disulfide bonding between molecules. Then, the proinsulin fusion protein is treated with cyanogen bromide (hereinafter referred to as ‘CNBr’) to cleave methionine residue connecting the leader peptide with proinsulin. After completion of the cleavage, CNBr is removed and the resulting proinsulin is separated, purified, and refolded with an oxidation and reduction system. Proinsulin is converted into active insulin by removing C-chain between A-chain and B-chain using trypsin and carboxypeptidase B. Insulin is purified by ion exchange chromatography and reverse phase high performance chromatography and zinc-crystallized in the final step.

The above-described method includes complex purification processes, and thus the conversion of the proinsulin fusion protein into insulin has a low yield and has problems requiring considerable expenses and time in terms of industrial production.

Also, though the expression level of the fusion protein may be increased by the above-described method, the final yield of the recombinant human insulin does not reach a satisfactory level (Goeddel D V, et al. Proc Natl Acad Sci USA. 1979;76:106-10, Talmadge K, et al. Proc Natl Acad Sci USA. 1980;77:3988-92, Sung W L, et al. Proc Natl Acad Sci USA. 1986;83:561-5).

Further, in terms of industrial production, the use of toxic CNBr is attended with danger in handling a toxic substance and brings about problems associated with much expense required to dispose of the used CNBr. Therefore, the leader peptide is preferably cleaved by a protease.

As enzymatic cleavage methods, the following have been developed.

Evans et al. fused a peptide comprising 8 amino acids, containing a metal binding site, and a renin cleavage site to the N-terminal of a target protein to cleave the leader peptide with renin as a protease (Evans D B, et al. Protein Expr Purif. 1991;2:205-13).

Sharma et al. used a peptide comprising 9 amino acids, containing 6 successive histidines, and a renin cleavage site as a leader peptide (Sharma S K, et al. Biotechnol Appl Biochem. 1991; 14:69-81).

For production of insulin, a method is developed, in which a proinsulin precursor having a recognition site which can be cleaved by a protease is expressed in E. coli, and the obtained inclusion bodies are subjected to refolding and other purification process.

For example, U.S. Pat. Nos. 5,126,249 and 5,378,613 disclose a method for preparing a gene encoding methionine-tyrosine or arginine-proinsulin by inserting only one amino acid between methionine, left only at the translational initiation site in E. coli, and the target protein. Generally, expression of a non-fusion protein results in a low level or the product is readily degraded, and the transcription and the translation may be damaged. But, it is possible to obtain a high expression level by this method.

However, in this method, cathepsin C or dipeptidyl-aminopeptidase should be used to remove two amino acids in front of proinsulin prior to the cleavage of C-chain by a protease. Consequently, an additional enzymatic reaction should be further included, complicating the purification process.

As another example, U.S. Pat. Nos. 5,227,293 and 5,358,857 disclose methods for expressing a protein comprising methionine as a translational initiation site, a peptide encoded by a short nucleotide sequence of (DCD)x, an enzyme cleavage site and a proinsulin analogue, which are sequentially fused together, in a microorganism. In the (DCD)x sequence, D represents adenine, guanine or thymine, C represents cytosine, and x represents 4 to 12. Therefore, amino acids encoded by the sequence are limited to serine, threonine or alanine. In this method, intact proinsulin is not used as a target protein and the mini-proinsulin having the C-chain composed of only one arginine is fused to the leader peptide.

However, in the above patent, there is no description of an example to convert the proinsulin fusion protein into insulin using trypsin and carboxypeptidase B simultaneously, and thus it is not considered that the complexity of the purification process is solved.

Also, Chen et al. expressed methionine-lysine-proinsulin composite in E. coli, thereby improving the expression level and simplifying the purification process (Chen J Q, et al. Appl Biochem Biotechnol. 1995;55:5-15).

However, the method has problems in that a large amount of insulin by-products are generated when the methionine-lysine-proinsulin is cleaved with trypsin and carboxypeptidase B to produce active insulin (Yang Z H, et al. Appl Biochem Biotechnol. 1999;76:107-14).

Korean Patent Registration No. 1002029580000 discloses a method for improving the efficiency of refolding and facilitating enzymatic cleavage by expressing a leader peptide-proinsulin composite. In this method, the leader peptide is composed of the N-terminal fragment of β-galactosidase, 6 successive threonines, and two amino acids comprising lysine or arginine. The leader peptide shows hydrophilic property as a whole, and thus it exerts a little influence on the refolding of proinsulin and a protease can readily recognize it and react.

However, in the above patent, there is no description of the generation of insulin by-products when the fusion protein is converted into human insulin through enzymatic cleavage, and thus it is not sure whether the problems associated with the generation of the by-products are solved. Indeed, it has been shown that a large amount of insulin by-products is generated upon conversion into insulin. Also, since the efficiency of the enzymatic cleavage is low, though the expression level of the fusion protein is high, the separation of insulin from the by-products in the subsequent processes becomes difficult. Consequently, the yield of the insulin production is low.

Meanwhile, Jonasson et al. succeeded in enzymatic cleavage with trypsin by expressing two IgG binding domain (hereinafter referred to as ‘ZZ’)-a linker comprising one or more amino acids of lysine or arginine-proinsulin composite (Jonasson P, et al. Eur J Biochem. 1996;236:656-61). In this method, proinsulin is refolded in the form of the ZZ leader peptide fused thereto, then the leader peptide and C-chain of proinsulin is concomitantly cleaved by trypsin and carboxypeptidase B, which simplifies the enzymatic treatment process.

However, in this method, the number of amino acids forming the ZZ leader peptide is greater than the number of amino acids forming proinsulin, and thus more than half polypeptide should be removed from the expressed recombinant protein in the purification process, which relatively reduces the yield. Also, the use of the lysine-arginine linker has a problem of the generation of a by-product with one arginine attached to B-chain of insulin.

As a similar example, U.S. Pat. No. 6,001,604 discloses a method for expressing SOD (superoxide dismutase)-arginine-proinsulin composite. In this method, the C-chain of the proinsulin comprises one or two amino acids, the proinsulin is refolded in the form of the SOD leader peptide fused thereto and the amino acids of the C-chain and the SOD are concomitantly cleaved by trypsin and carboxypeptidase B.

However, this method also has a problem in that the number of amino acids forming the leader peptide is greater than the number of amino acids forming the modified proinsulin.

U.S. Pat. No. 6,068,993 discloses a method for expressing a fusion protein of a leader peptide comprising 11 amino acids, containing 6 successive histidines and an arginine as the C-terminal amino acid, and proinsulin. In this method, the fusion protein is converted into insulin by enzymatic reaction after metal ion adsorption process and refolding. Then, the produced insulin is purified by ion exchange chromatography and reverse phase chromatography. According to this method, the chromatography using Ni-chelating Sepharose FF resin and the buffer solution exchange using Sephadex G-25 resin should be performed prior to the refolding, which makes the purification process complex.

Therefore, there are demands for a recombinant plasmid and a preparation method which can produce human insulin at a high yield in a simple process.

### SUMMARY OF THE INVENTION

In order to accomplish the above demands, it is an object of the present invention to provide recombinant plasmids which can stably express a fusion protein of a leader peptide and proinsulin or analogue thereof in microorganisms, in which the leader peptide has a site which can be selectively cleaved by an enzyme, and can be easily isolated from the target protein.

Also, in another aspect of the present invention, it is an object to provide a method for preparing insulin in a large amount by a simple process, in which a proinsulin fusion protein is converted into active insulin while minimizing generation of by-products, using the recombinant plasmid according to the present invention.

The plasmid according to the present invention comprises a sequence encoding a compound of the following formula (I):
R—B—X-A (I)

In the formula (I), R is a leader peptide represented by the following formula (II).
Met-Thr-Met-Ile-Thr-Y (II)

In the formula (II), Y is one selected from lysine, arginine, a peptide containing lysine as an amino acid at its C-terminal, or a peptide containing arginine as an amino acid at its C-terminal.

In the formula (I), B is human insulin B-chain or analogue thereof, X is a peptide connecting B with A, A is human insulin A-chain or analogue thereof.

In the plasmid according to the present invention, where Y is lysine, R of the formula (I) may be the following amino acid sequence.

| | |
|---|---|
| Met-Thr-Met-Ile-Thr-Lys: | SEQ ID NO. 1 |

In the plasmid according to the present invention, where Y is a peptide containing lysine as an amino acid at its C-terminal, R of the formula (I) may be the following amino acid sequences.

| | |
|---|---|
| Met-Thr-Met-Ile-Thr-Asp-Ser-Leu-Ala- | SEQ ID NO. 2 |
| Lys | |
| Met-Thr-Met-Ile-Thr-Asp-Ser-Leu-Ala- | SEQ ID NO. 3 |
| Val-Val-Leu-Gln-Lys | |
| Met-Thr-Met-Ile-Thr-Asp-Ser-Leu-Ala- | SEQ ID NO. 4 |
| Val-Val-Leu-Gln-Gly-Ser-Leu-Gln-Lys | |

In the plasmid according to the present invention, where Y is arginine, R of the formula (I) may be the following amino acid sequence.

| | |
|---|---|
| Met-Thr-Met-Ile-Thr-Arg | SEQ ID NO. 5 |

In the plasmid according to the present invention, where Y is a peptide containing argnine as an amino acid at its C-terminal, R of the formula (I) may be the following amino acid sequences.

| | |
|---|---|
| Met-Thr-Met-Ile-Thr-Asp-Ser-Leu-Ala- | SEQ ID NO. 6 |
| Arg | |
| Met-Thr-Met-Ile-Thr-Asp-Ser-Leu-Ala- | SEQ ID NO. 7 |
| Val-Val-Leu-Gln-Arg | |
| Met-Thr-Met-Ile-Thr-Asp-Ser-Leu-Ala- | SEQ ID NO. 8 |
| Val-Val-Leu-Gln-Gly-Ser-Leu-Gln-Arg | |

According to the present invention, preferred examples of nucleotide sequences encoding the amino acid sequences of SEQ ID NO. 1 to 8 are as follows.

| | |
|---|---|
| ATG ACC ATG ATT ACG AAG | SEQ ID NO. 9 |
| ATG ACC ATG ATT ACG GAT TCA CTG GCC | SEQ ID NO. 10 |
| AAG | |
| ATG ACC ATG ATT ACG GAT TCA CTG GCC | SEQ ID NO. 11 |
| GTC GTT TTA CAA AAG | |
| ATG ACC ATG ATT ACG GAT TCA CTG GCA | SEQ ID NO. 12 |
| GTC GTT TTA CAA GGT TCT CTG CAG AAG | |
| ATG ACC ATG ATT ACG CGT | SEQ ID NO. 13 |
| ATG ACC ATG ATT ACG GAT TCA CTG GCC | SEQ ID NO. 14 |
| CGT | |
| ATG ACC ATG ATT ACG GAT TCA CTG GCC | SEQ ID NO. 15 |
| GTC GTT TTA CAA CGT | |
| ATG ACC ATG ATT ACG GAT TCA CTG GCA | SEQ ID NO. 16 |
| GTC GTT TTA CAA GGT TCT CTG CAG GGT | |

The leader peptide according to the present invention acts as a mask to help proinsulin or analogue thereof stably exist and be expressed since it can be stably expressed in E. coli. In the present invention, the short leader peptide is used, and thus the ratio of the target protein to the leader peptide is relatively high and the target protein is readily isolated and purified by cleaving the fusion protein.

Also, in the leader peptide according to the present invention, lysine or arginine at C-terminal provides a site which can be selectively cleaved by trypsin. Therefore, it is possible to convert the proinsulin fusion protein into active insulin by enzymatic cleavage without toxic CNBr treatment which has been conventionally used.

The plasmid according to the present invention has a sequence encoding proinsulin or analogue thereof (B—X—Y) connected with 3′-end of the above sequence.

Representative example of the proinsulin analogue according to the present invention is a protein having positions of residue No. 28 and residue No. 29 of B-chain exchanged with each other (hereinafter referred to as ‘LysB₂₈ProB₂₉ analogue’). The LysB₂₈ProB₂₉ insulin analogue has effect equal to that of human insulin and can be more rapidly absorbed from a subcutaneous injection site.

In the preparation of the plasmid according to the present invention, the gene encoding the proinsulin fusion protein is obtained by cleaving pPRO plasmid (Korean Patent Registration No. 1000766010000) with restriction enzymes of EcoRI and Bg1II, ligating the product with ligase to construct pHHI plasmid, and performing Polymerase Chain Reaction (PCR) using the resulting plasmid as a template.

The pHHI plasmid used in the present invention has a gene at 3′-end of tac promoter. The gene encodes a fusion protein which is sequentially expressed in the order of a peptide comprising 28 amino acids, containing a histidine tag, a methionine, and proinsulin.

In the preparation of the plasmid according to the present invention, expression vectors used in cloning include any vectors which can show a high expression level in E. coli. Preferred examples include pET-24a(+) vector containing strong T7 promoter (Novagen, Catalogue No. 69749-3) and pHHI-derived vectors containing E. coli rrnB P2 promoter (Lukacsovich T, et al. Gene. 1989;78:189-94) or rac promoter (Boros I, et al. Gene. 1986;42:97-100).

Representative examples of the plasmid according to the present invention include pK-BKpi type, pK-BRpi type, pPT-BKpi type, pPT-BRpi type, pPL-BKpi type, pPL-BRpi type, pPLD-BKpi type, pPLD-BRpi type, pPT-BKpiKP type, and pPT-BRpiKP type plasmids, which are classified by vectors, peptide types and target proteins to be expressed.

### BRIEF DESCRIPTION OF THE DRAWINGS

The above objects, features and advantages of the present invention will become more apparent from the following detailed description when taken in conjunction with the accompanying drawings, in which:

FIG. 1 is a view schematically showing the structures of pK-BKpi type and pK-BRpi type plasmids according to the present invention and the preparation method thereof;

FIG. 2 is a view schematically showing the structure of pPT vector and the preparation method thereof;

FIG. 3 is a view schematically showing the structures of pPT-BKpi type and pPT-BRpi type plasmids according to the present invention and the preparation method thereof;

FIG. 4 is a view schematically showing the structures of pPL-BKpi type, pPL-BRpi type, pPLD-BKpi type and pPLD-BRpi type plasmids according to the present invention and the preparation method thereof;

FIG. 5 is a view schematically showing the structure of the leader peptides in the proinsulin fusion proteins expressed by the plasmids according to the present invention;

FIG. 6 is a view showing the result of the comparison of the fusion protein expression level by the pK-BKpi type plasmids according to the present invention with the methionine-lysine-proinsulin fusion protein expression plasmid used before;

FIG. 7 is a view showing the result of the comparison of the fusion protein expression level by pK-B5Kpi, pPT-B5Kpi, pPL-B5Kpi and pPLD-B5Kpi plasmids according to the present invention; and

FIG. 8 is a view showing the result of the comparison of the by-products generation by the plasmid according to the present invention with that of the methionine-lysine-proinsulin fusion protein expression plasmid used before.

### DETAILED DESCRIPTION OF THE PREFERRED EMBODIMENT

Now, the foregoing plasmids according to the present invention are explained concretely with reference to drawings.

1) pK-BKpi Type and pK-BRpi Type Plasmids

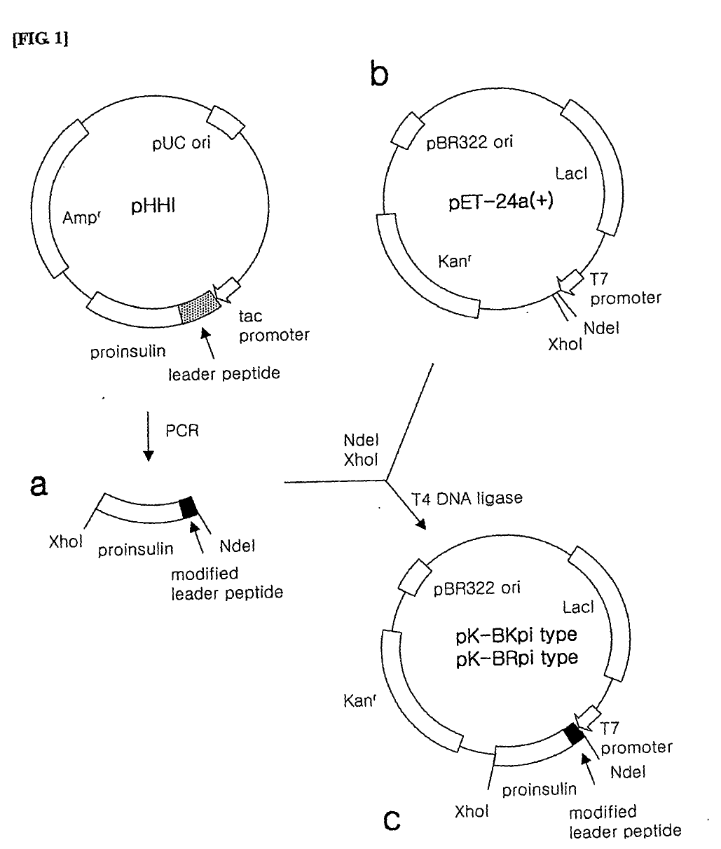

As shown in FIG. 1, the pK-BKpi type and pK-BRpi type plasmids are recombinant plasmids (FIG. 1c) prepared by synthesizing the proinsulin fusion protein gene (FIG. 1a) through PCR using pHHI plasmid as a template and ligating the product to expression vector pET-24a(+) (FIG. 1b) which has been cleaved with proper restriction enzymes.

The pK-BKpi type plasmids are classified into pK-B5Kpi, pK-B9Kpi and pK-B13Kpi according to their leader peptide types, which can express the fusion proteins of the leader peptides of SEQ ID NO. 1, 2 and 3, respectively, with proinsulin.

The pK-BRpi type plasmids are classified into pK-B5Rpi, pK-B9Rpi and pK-B13Rpi according to their leader peptide types, which can express the fusion proteins of the leader peptides of SEQ ID NO. 5, 6 and 7, respectively, with proinsulin.

2) pPT-BKpi Type and pPT-BRpi Type Plasmids

The pPT-BKpi type and pPT-BRpi type plasmids are plasmids prepared by constructing the pPT vector and using it as a backbone.

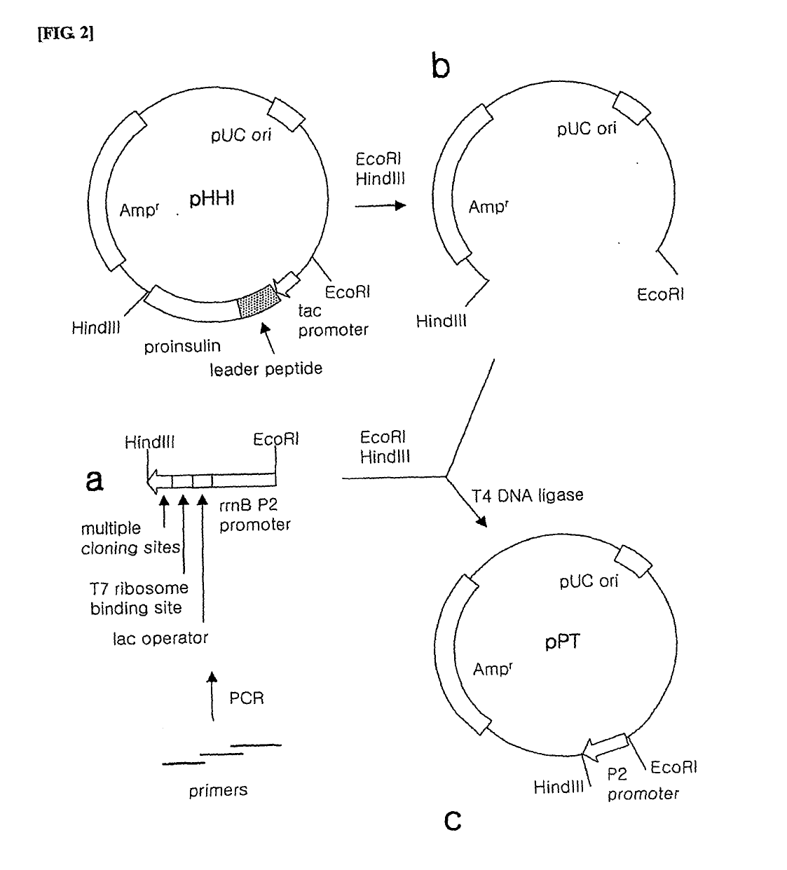

As shown in FIG. 2, the pPT vector is the recombinant vector (FIG. 2c) prepared by synthesizing the rrnB P2 promoter (FIG. 2a) through PCR, cleaving pHHI plasmid with proper restriction enzymes and ligating the PCR product to the vector with tac promoter and the proinsulin fusion protein gene removed (FIG. 2b).

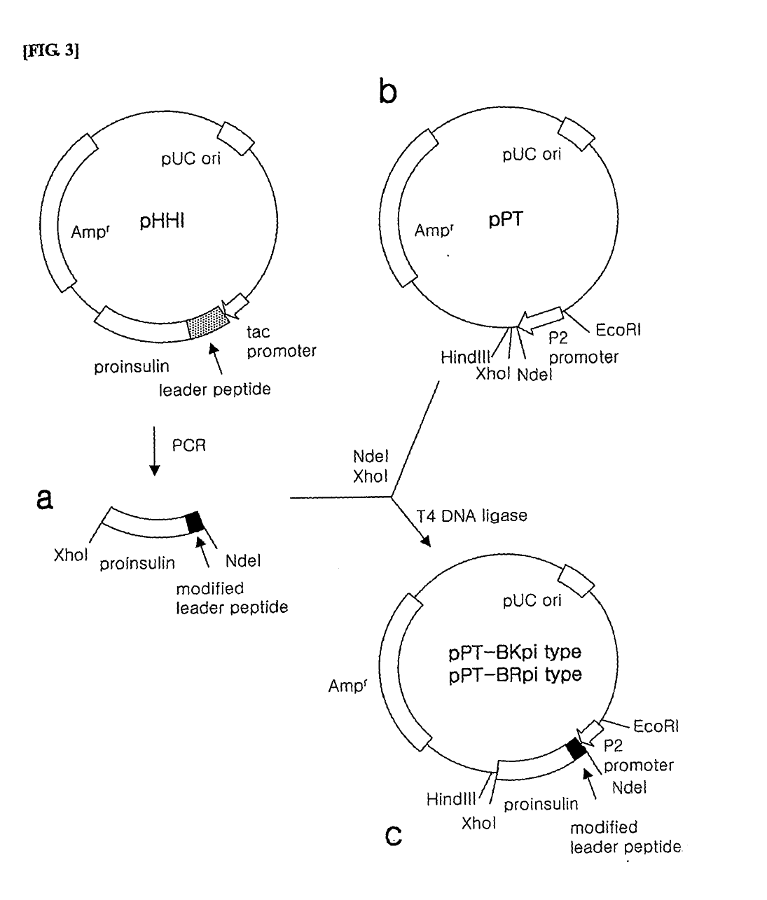

As shown in FIG. 3, the pPT-BKpi type and pPT-BRpi type plasmids are recombinant plasmids (FIG. 3c) prepared by synthesizing the proinsulin fusion protein gene (FIG. 3a) through PCR using pHHI plasmid as a template and ligating the product to the pPT expression vector (FIG. 3b) which has been cleaved with proper restriction enzymes.

The pPT-BKpi type plasmids are classified into pPT-B5Kpi, pPT-B9Kpi and pPT-B13Kpi according to their leader peptide types, which can express the fusion proteins of the leader peptides of SEQ ID NO. 1, 2 and 3, respectively, with proinsulin.

The pPT-BRpi type plasmids are classified into pPT-B5Rpi, pPT-B9Rpi and pPT-B13Rpi according to their leader peptide types, which can express the fusion proteins of the leader peptides of SEQ ID NO. 5, 6 and 7, respectively, with proinsulin.

3) pPT-17Kpi and pPT-17Rpi Plasmids

The pPT-17Kpi and pPT-17Rpi plasmids are recombinant plasmids prepared by synthesizing the proinsulin fusion protein gene through PCR using pHHI plasmid as a template and ligating the product to the pPT expression vector which has been cleaved with proper restriction enzymes.

The pPT-17Kpi plasmids express the fusion protein of the leader peptide of SEQ ID NO. 4 with proinsulin.

The pPT-17Rpi plasmids express the fusion protein of the leader peptide of SEQ ID NO. 8 with proinsulin.

4) pPL-BKpi Type, pPL-BRpi Type, pPLD-BKpi Type and pPLD-BRpi Type Plasmids

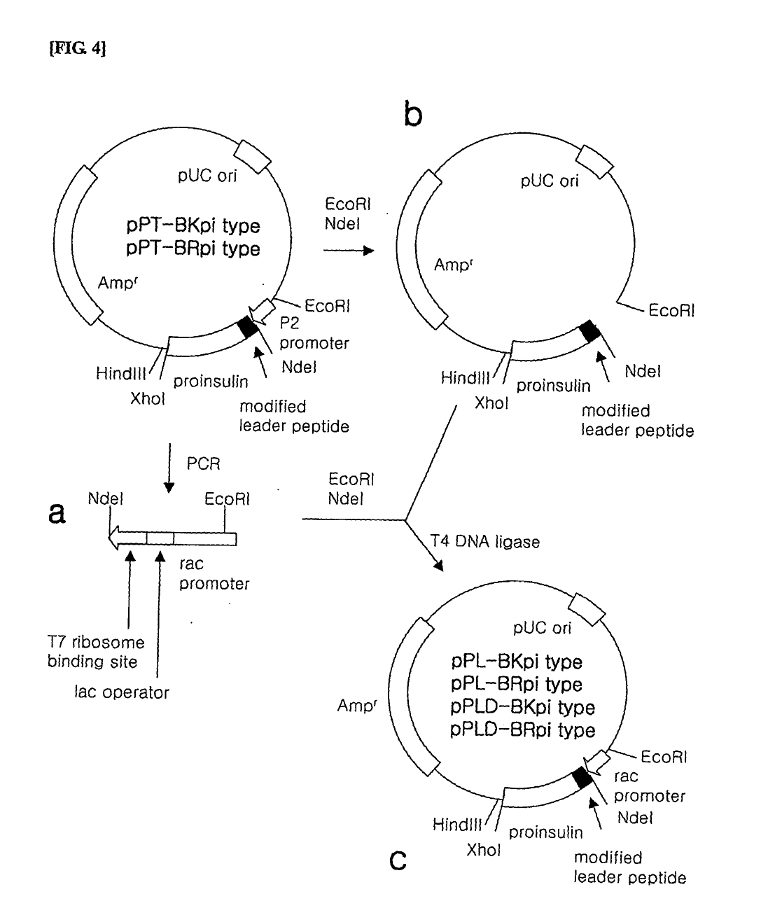

As shown in FIG. 4, the pPL-BKpi type, pPL-BRpi type, pPLD-BKpi type and pPLD-BRpi type plasmids are recombinant plasmids (FIG. 4c) prepared by synthesizing the rac promoter (FIG. 4a) through PCR using pPT-BKpi type or pPT-BRpi type plasmids as a template and ligating the product to pPT-BKpi type or pPT-BRpi type plasmids (FIG. 4b) with P2 promoter removed by restriction enzyme cleavage.

The pPL-BKpi type plasmids are classified into pPL-B5Kpi, pPL-B9Kpi and pPL-B13Kpi according to their leader peptide types, which express the fusion proteins of the leader peptides of SEQ ID NO. 1, 2 and 3, respectively, with proinsulin.

The pPL-BRpi type plasmids are classified into pPL-B5Rpi, pPL-B9Rpi and pPL-B 13Rpi according to their leader peptide types, which express the fusion proteins of the leader peptides of SEQ ID NO. 5, 6 and 7, respectively, with proinsulin.

The pPID-BKpi type plasmids are classified into pPLD-B5Kpi, pPLD-B9Kpi and pPLD-B13Kpi according to their leader peptide types, which express the fusion proteins of the leader peptides of SEQ ID NO. 1, 2 and 3, respectively, with proinsulin.

The pPLD-BRpi type plasmids are classified into pPLD-B5Rpi, pPLD-B9Rpi and pPLD-B13Rpi according to their leader peptide types, which express the fusion proteins of the leader peptides of SEQ ID NO. 5, 6 and 7, respectively, with proinsulin.

5) pPT-BKpiKP Type and pPT-BRpiKP Type Plasmids

The pPT-BKpiKP type and pPT-BRpiKP type plasmids are prepared by synthesizing the desired gene encoding LysB₂₈ProB₂₉ analogue fusion protein from pPT-BKpi type and pPT-BRpi type plasmids by site-directed mutagenesis through PCR and ligating the product to pHHI plasmid which has been cleaved with proper restriction enzymes.

The pPT-BKpiKP type plasmids are classified into pPT-B5KpiKP, pPT-B9KpiKP and pPT-B13KpiKP according to their leader peptide types, which express the fusion proteins of the leader peptides of SEQ ID NO. 1, 2 and 3, respectively, with the LysB₂₈ProB₂₉ analogue.

The pPT-BRpiKP type plasmids are classified into pPT-B5RpiKP, pPT-B9RpiKP and pPT-B13RpiKP according to their leader peptide types, which express the fusion proteins of the leader peptides of SEQ ID NO. 5, 6 and 7, respectively, with the LysB₂₈ProB₂₉ analogue.

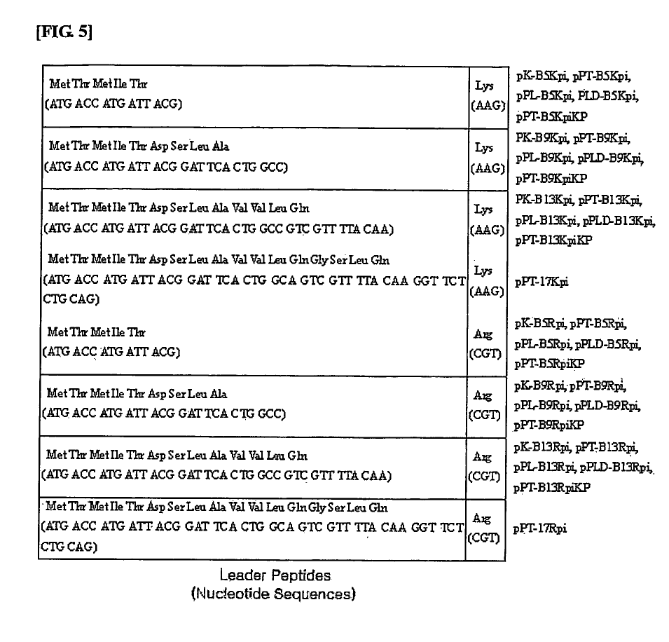

The structures of the plasmids prepared as described above are shown in FIG. 5.

The plasmid according to the present invention can stably express the proinsulin fusion protein which can be enzymatically cleaved for conversion into active insulin in a simple method while generating a very small amount of by-products in the enzymatic cleavage.

Accordingly, considering the above requirements collectively, the plasmid according to the present invention can produce insulin at a high yield.

The method for preparing insulin using the plasmid according to the present invention comprises: (a) a step to induce the expression of a compound of the formula (I) from a microorganism containing the plasmid according to the present invention, (b) a step of cell disruption and dissolution, (c) a step of refolding, (d) a step of co-cleavage of R and X by an enzymatic reaction, and (e) a step of purification of active insulin by chromatography.

In the (a) step, a proper microorganism is transformed with the plasmid according to the present invention. Strains which can be preferably used for the transformation in the present invention include E. coli BL21(DE3) for pK-B5Kpi plasmid and E. coli JM109 for pPT-B5Kpi plasmid.

A fed batch fermentation is conducted for high cell density culture (HCDC) of the transformed microorganism in a large quantity. Conditions for the fermentation are as follows; the temperature is maintained at 37° C., the dissolved oxygen is maintained at 30% air saturation, the ventilation rate is 1 VVM, and the pH is maintained at 6.8 to 7.0. When the microorganism propagates to a proper concentration, IPTG (Isopropyl β-thiogalactopyranoside) is added to induce protein expression.

In the (b) step, the cells, obtained by the fermentation as described above, are suspended in a buffer solution, disrupted and centrifuged to separate inclusion bodies, which are then washed and dissolved in a urea solution.

In this step, a sulfonation process may be performed with the washed inclusion bodies, if necessary. In this case, the inclusion bodies are converted into the S-sulfonated form of the proinsulin fusion protein, and then centrifuged to remove precipitates.

In the (c) step, the resulting supernatant is diluted in purified water, followed by deaeration and sealing. β-mercaptoethanol is added to a glycine buffer solution contained separately, followed by sealing. Two solutions are rapidly mixed for the refolding of the protein.

In the (d) step, the leader peptide and C-chain are concomitantly removed from the refolded proinsulin fusion protein using trypsin and carboxypeptidase B to form active insulin.

Optimal conditions for this step include pH 7 to 8, a reaction temperature of 4° C. to 28° C., a trypsin level of 0.1 u to 0.5 u per protein 1 mg, a carboxypeptidase B level of 0.1 u to 0.3 u per protein 1 mg and a reaction time of 12 to 24 hours.

Also, in this step, enzymes immobilized on a suitable resin may be used as needed. For example, a combination of immobilized trypsin and immobilized carboxypeptidase B may be used.

In the (e) step, the active insulin is finally purified by ion exchange and reverse phase high pressure liquid chromatography.

The method for producing insulin using the plasmid according to the present invention directly performs the refolding by rapidly mixing the proinsulin solution and the glycine buffer solution without chromatography process, which is conventionally performed for refolding, and thus solves the problems related with waste water owing to use of an organic solvent and resin washing solution, and improves the efficiency of refolding in a simple process.

Also, the method for producing insulin using the plasmid according to the present invention simplifies the conversion of the proinsulin fusion protein into active insulin in a single process by the structural features of the plasmid according to the present invention, thereby increasing the efficiency of the process, and provides an environmentally friendly process by solving the problems associated with the use of toxic formic acid or CNBr which has been conventionally used. Further, since the generation of by-products after the conversion into active insulin is minimized, the final insulin yield is maximized.

Therefore, the plasmid according to the present invention and the method for producing insulin using the same may be applied to industrial mass-production of human insulin and be usefully used in various fields needing insulin, such as treatment of diabetes, including preparation of pharmaceutical composition containing insulin as an effective ingredient.

Now, the present invention will be explained through the following examples. However, the present invention is not limited thereto.

### EXAMPLE 1

### Preparation of Inventive pK-BKpi Type and pK-BRpi Type Plasmids

The pK-BKpi type and pK-BRpi type plasmids were prepared as follows.

1) Preparation of Proinsulin Fusion Protein Gene

In order to prepare the proinsulin fusion protein gene to be inserted into the expression vector, PCR was performed using pHHI, the expression plasmid of the proinsulin fusion protein as a template.

Here, a forward primer among the used primers was synthesized to include a NdeI restriction enzyme recognition site, a sequence encoding the leader peptides of SEQ ID NO. 1, 2, 3, 5, 6 or 7 and a sequence encoding the N-terminal fragment of insulin B-chain in order (the leader peptide of SEQ ID NO. 1: SEQ ID NO. 17, the leader peptide of SEQ ID NO. 2: SEQ ID NO. 18, the leader peptide of SEQ ID NO. 3: SEQ ID NO. 19, the leader peptide of SEQ ID NO. 5: SEQ ID NO. 20, the leader peptide of SEQ ID NO. 6: SEQ ID NO. 21, the leader peptide of SEQ ID NO. 7: SEQ ID NO. 22), while a reverse primer was synthesized to include a XhoI restriction enzyme recognition site. (SEQ ID NO. 23). The sequences of the respective primers are as follows.

| | |
|---|---|
| 5′- CAC CAG CAT ATG ACC ATG ATT ACG | SEQ ID NO. 17 |
| AAG TTT GTG AAC CAA CAC CTG T -3′ | |
| 5′- CAC CAG CAT ATG ACC ATG ATT ACG | SEQ ID NO. 18 |
| GAT TCA CTG GCC AAG TTT GTG AAC CAA | |
| CAC CTG TGC -3′ | |
| 5′- CAC CAG CAT ATG ACC ATG ATT ACG | SEQ ID NO. 19 |
| GAT TCA CTG GCC GTC GTT TTA CAA AAG | |
| TTT GTG AAC CAA CAC CTG TGC -3′ | |
| 5′- CAC CAG CAT ATG ACC ATG ATT ACG | SEQ ID NO. 20 |
| CGT TTT GTG AAC CAA CAC CTG T-3′ | |
| 5′- CAC CAG CAT ATG ACC ATG ATT ACG | SEQ ID NO. 21 |
| GAT TCA CTG GCC CGT TTT GTG AAC CAA | |
| CAC CTG TGC -3′ | |
| 5′- CAC CAG CAT ATG ACC ATG ATT ACG | SEQ ID NO. 22 |
| GAT TCA CTG GCC GTC GTT TTA CAA CGT | |
| TTT GTG AAC CAA CAC CTG TGC -3′ | |
| 5′- GCA TGC CTC GAG GTC GAC TCT | SEQ ID NO. 23 |
| AGA -3′ | |

During PCR, the denaturation was performed for 30 seconds at 94° C., the annealing reaction was performed for 30 seconds at 55° C. and the polymerization was performed for 25 seconds at 72° C. The above cycle was repeated 30 times. DNA obtained from the PCR was cleaved with restriction enzymes NdeI (Takara, Japan) and XhoI (Gibco, U.S.), electrophoresed on 1% agarose gel to isolate a gene segment of 0.3 kbp.

2) Preparation of Expression Vector

pET-24a(+) vector as the expression vector was cleaved with restriction enzymes NdeI and XhoI, electrophoresed on 1% agarose gel to isolate DNA segment of 5.2 Kb.

3) Cloning

The two DNA segments prepared as above were joined to each other using T4 DNA ligase (Takara, Japan) to form the plasmid. E. coli BL21(DE3) was transformed with each of the prepared plasmids by the calcium chloride method. The transformed cells resistant to kanamycin were selected. The plasmid DNA was isolated from each transformant and confirmed that the desired DNA had been properly inserted using an analysis by restriction enzyme cleavage.

The pK-B5Kpi of the plasmids according to the present invention was deposited in Korea Research Institute of Bioscience and Biotechnology Gene Bank on Nov. 4, 2002 under the accession No. KCTC 10363BP.

### EXAMPLE 2

### Preparation of Inventive pPT-BKpi Type and pPT-BRpi Type Plasmids

The pPT-BKpi type and pPT-BRpi type plasmids according to the present invention were prepared as follows.

1) Preparation of Promoter

In order to prepare a P2 promoter, a lac operator, a T7 ribosome binding site and restriction enzyme cleavage sites to be inserted into the vector, PCR was performed using tree primers including a part of the sequence.

The first primer was synthesized to have an EcoRI restriction enzyme recognition site and the upstream of P2 promoter in the forward direction (SEQ ID NO. 24), the second primer was synthesized to have −35 region, −10 region of P2 promoter and lac operator sequentially in the reverse direction (SEQ ID NO. 25) and the third primer was synthesized to have a T7 ribosome binding site and NdeI, KpnI, XhoI, SalI, HindIII restriction enzyme cleavage sites sequentially in the reverse direction (SEQ ID NO. 26).

Since the 3′-end of the first primer and the 3′-end of the second primer had 18 complementary bases, and the 5′-end of the second primer and the 3 ′-end of the third primer had 18 identical bases, the three primers could be joined together by PCR. The sequences of the primers are shown below.

| | |
|---|---|
| 5′- CAT GTT GAA TTC TGC GCC ACC ACT | SEQ ID NO. 24 |
| GAC ACG GAC AAC GGC AAA CAC GCC GCC | |
| GGG TCA GCG GGG TTC TCC TGA GAA CTC | |
| CGG CAG AGA AAG C -3′ | |
| 5′- TGT TTC CTG TGT GAA ATT GTT ATC | SEQ ID NO. 25 |
| CGC TCA CAA TTC CAT AAT ACG CCT TCC | |
| CGC TAC AGA GTC AAG CAT TTA TTT TTG | |
| CTT TCT CTG CCG GAG TTC -3′ | |
| 5′- ACA GCC AAG CTT GTC GAC TCG AGG | SEQ ID NO. 26 |
| TAC CGA CAT ATG TAT ATC TCC TTC TTA | |
| AAG TTA AAC AAA ATT ATT TCT AGA AGC | |
| TGT TTC CTG TGT GAA ATT -3′ | |

During PCR, the denaturation was performed for 30 seconds at 94° C., the annealing reaction was performed for 30 seconds at 55° C. and the polymerization was performed for 20 seconds at 72° C. The above cycle was repeated 30 times.

2) Preparation of pPT Vector

The promoter DNA amplified by the PCR was cleaved with restriction enzymes EcoRI (Takara, Japan) and HindIII (Gibco, U.S.) and electrophoresed on 1% agarose gel to isolate a DNA segment of 0.2 kbp.

The pHHI plasmid was cleaved with restriction enzymes EcoRI and HindIII and electrophoresed on 1% agarose gel to isolate a DNA segment of 3.1 kbp with tac promoter and the proinsulin fusion protein gene removed.

The two DNA segments prepared as above were joined together using T4 DNA ligase to construct the vector. E. coli JM109 was transformed with the vector by the calcium chloride method. The transformed cells resistant to ampicillin were selected. The vector DNA was isolated from each transformant and confirmed that the desired DNA had been properly inserted using an analysis by restriction enzyme cleavage.

3) Preparation of pPT-BKpi Type and pPT-BRpi Type Plasmids

Using the same method as in the preparation of pK-BKpi type and pK-BRpi type plasmids, the proinsulin fusion protein gene was obtained from pHHI plasmid by PCR, cleaved with restriction enzymes NdeI and XhoI and electrophoresed on 1% agarose gel to isolate a gene segment of 0.3 kbp.

The pPT vector was cleaved with restriction enzymes NdeI and XhoI and electrophoresed on 1% agarose gel to isolate a DNA segment of 3.2 kbp.

The two DNA segments prepared as above were joined together using T4 DNA ligase to produce the plasmid. E. coli JM109 was transformed with the produced plasmid by the calcium chloride method. The transformed cells resistant to ampicillin were selected. The plasmid DNA was isolated from each transformant and confirmed that the desired DNA had been properly inserted using an analysis by restriction enzyme cleavage.

### EXAMPLE 3

### Preparation of Inventive pPT-17Kpi and pPT-17Rpi Plasmids

The pPT-17Kpi and pPT-17Rpi plasmids were prepared as follows.

1) Preparation of Proinsulin Fusion Protein Gene

In order to prepare the proinsulin fusion protein gene to be inserted into the expression vector, PCR was performed using pHHI, the expression plasmid of the proinsulin fusion protein as a template.

Here, a forward primer among the used primers was synthesized to include a NdeI restriction enzyme recognition site, a sequence encoding the leader peptides of SEQ ID NO. 4 or 8 and a sequence encoding the N-terminal fragment of insulin B-chain in order (the leader peptide of SEQ ID NO. 4: SEQ ID NO. 27, the leader peptide of SEQ ID NO. 8: SEQ ID NO. 28), while a reverse primer was synthesized to include a XhoI restriction enzyme recognition site. (SEQ ID NO. 23). The sequences of the respective primers are as follows.

| | |
|---|---|
| 5′- GAA ACA CAT ATG ACC ATG ATT ACG | SEQ ID NO. 27 |
| GAT TCA CTG GCA GTC GTT TTA CAA GGT | |
| TCT CTG CAG AAG TTT GTG AAC CAA CAC | |
| CTG TG -3′ | |
| 5′- GAA ACA CAT ATG ACC ATG ATT ACG | SEQ ID NO. 28 |
| GAT TCA CTG GCA GTC GTT TTA CAA GGT | |
| TCT CTG CAG CGT TTT GTG AAC CAA | |
| CAC CTG TG -3′ | |

During PCR, the denaturation was performed for 30 seconds at 94° C., the annealing reaction was performed for 30 seconds at 55° C. and the polymerization was performed for 25 seconds at 72° C. The above cycle was repeated 30 times. DNA obtained from the PCR was cleaved with restriction enzymes NdeI and XhoI, electrophoresed on 1% agarose gel to isolate a gene segment of 0.3 kbp.

2) Preparation of Expression Vector

pPT vector as an expression vector was cleaved with restriction enzymes NdeI and XhoI, electrophoresed on 1% agarose gel to isolate DNA segment of 3.2 Kb.

3) Cloning

The two DNA segments prepared as above were joined to each other using T4 DNA ligase (Takara, Japan) to form the plasmid. E. coli JM109 was transformed with each of the prepared plasmids by the calcium chloride method. The transformed cells resistant to ampicillin were selected. The plasmid DNA was isolated from each transformant and confirmed that the desired DNA had been properly inserted using an analysis by restriction enzyme cleavage.

### EXAMPLE 4

### Preparation of Inventive pPL-BKpi Type, pPL-BRpi Type, pPLD-BKpi Type and pPLD-BRpi Type Plasmids

The pPL-BKpi type, pPL-BRpi type, pPLD-BKpi type and pPLD-BRpi type plasmids were prepared as follows.

1) Preparation of Promoter

In order to prepare a rac promoter, a lac operator, a T7 ribosome binding site and a restriction enzyme cleavage site to be inserted into the plasmid, PCR was performed using pPT-B5Kpi plasmid as a template.

Among the used three primers, the first primer was complementary to about 60 bp upstream from the EcoRI restriction enzyme recognition site of the pPT-B5Kpi plasmid in the forward direction (SEQ ID NO. 29), the second primer was synthesized to have −35 region of P2 promoter, −10 region of lac promoter and a part of lac operator sequentially in the reverse direction (pPL vector: SEQ ID NO. 30, pPLD vector: SEQ ID NO. 31), the third primer was synthesized to have lac operator, T7 ribosome binding site and NdeI restriction enzyme cleavage site sequentially in the reverse direction (SEQ ID NO. 32). The 5′-end of the second primer and the 3′-end of the third primer had 18 identical bases. The sequences of the primers are as follows.

| | |
|---|---|
| 5′- AGT AAG GCA ACC CCG CCA GC -3′ | SEQ ID NO. 29 |
| 5′- TTA TCC GCT CAC AAT TCC ACA | SEQ ID NO. 30 |
| CAA CAT ACG AGC CTT CCC GCT ACA | |
| GAG T -3′ | |
| 5′- TTA TCC GCT CAC AAT TCC AAC ATA | SEQ ID NO. 31 |
| CGA GCC TTC CCG CTA CAG AGT -3′ | |
| 5′- TAG CGA CAT ATG TAT ATC TCG TTC | SEQ ID NO. 32 |
| TTA AAG TTA AAC AAA ATT ATT TCT AGA | |
| GGG AAA TTG TTA TCC GCT CAC AAT | |
| TCG-3′ | |

During PCR, the denaturation was performed for 30 seconds at 94° C., the annealing reaction was performed for 30 seconds at 55° C. and the polymerization was performed for 20 seconds at 72° C. The above cycle was repeated 30 times.

2) Preparation of pPL-BKpi Type, pPL-BRpi Type, pPLD-BKpi Type and pPLD-BRpi Type Plasmids

The promoter DNA amplified by the PCR was cleaved with restriction enzymes EcoRI and NdeI and electrophoresed on 1% agarose gel to isolate a DNA segment of 0.2 kbp.

The pPT-BKpi type or pPT-BRpi type plasmid was cleaved with restriction enzymes EcoRI and NdeI and electrophoresed on 1% agarose gel to isolate a DNA segment of 3.4 kbp with P2 promoter removed.

The two DNA segments prepared as above were joined together using T4 DNA ligase to produce the plasmid. E. coli JM109 was transformed with the produced plasmid by the calcium chloride method. The transformed cells resistant to ampicillin were selected. The vector DNA was isolated from each transformant and confirmed that the desired DNA had been properly inserted using an analysis by restriction enzyme cleavage.

### EXAMPLE 5

### Preparation of Inventive pPT-BKpiKP Type and pPT-BRpiKP Type Plasmids

The pPT-BKpiKP type and pPT-BRpiKP type plasmids were prepared as follows.

1) Preparation of Proinsulin Analogue Fusion Protein Gene

In order to obtain the proinsulin analogue fusion protein gene, site-directed mutagenesis was performed by PCR using pPT-BKpi type or pPT-BRpi type plasmids as a template and the residue No. 28 and the residue No. 29 of proinsulin B-chain were exchanged with each other.

By PCR, a gene encoding P2 promoter and the leader peptide of SEQ ID NO. 1, 2, 3, 5, 6 or 7, B-chain, and the N-terminal fragment of C-chain was amplified.

Among the used primers, the first primer was complementary to about 60 bp upstream from the EcoRI restriction enzyme recognition site of pPT-BKpi type or pPT-BRpi type plasmids in the forward direction (SEQ ID NO. 29) and the second primer was synthesized to include a sequence with the residues Nos. 28 and 29 of B-chain exchanged with each other (SEQ ID NO. 33). The sequence of the primer is as follows.

| | |
|---|---|
| 5′- CTC CCG GCG GGT GGG CTT TGT | SEQ ID NO. 33 |
| GTA GAA GAA GCC -3′ | |

A gene encoding the C-terminal fragment of B-chain, C-chain and A-chain was amplified by PCR. Here, among the used primers, the first primer included a sequence with the residues Nos. 28 and 29 of B-chain exchanged with each other in the forward direction and was complementary to the primer of SEQ ID NO. 25 (SEQ ID NO. 34) and the second primer was complementary to about 80 bp downstream from the HindIII restriction enzyme recognition site of pPT-BKpi type or pPT-BRpi type plasmids in the reverse direction (SEQ ID NO. 35). The sequences of the primers are as follows.

| | |
|---|---|
| 5′- GGC TTC TTC TAC ACA AAG CCC ACC | SEQ ID NO. 34 |
| CGC CGG GAG -3′: | |
| 5′- CTG CCG CCA GGC AAA TTC TG -3′: | SEQ ID NO. 35 |

Since the two DNA segments have the same sequence encoding the C-terminal fragment of B-chain and the N-terminal fragment C-chain, PCR was performed using primers of SEQ ID NO. 29 and SEQ ID NO. 35 to form DNA comprising from P2 promoter to A-chain. As a result, a DNA which has P2 promoter and a gene encoding the leader peptide of SEQ ID NO. 1, 2, 3, 5, 6 or 7 and the proinsulin analogue with the residues B₂₈ and B₂₉ exchanged with each other was obtained.

The DNA was cleaved with restriction enzymes EcoRI and HindIII and electrophoresed on 1% agarose gel to isolate a gene segment of 0.5 kbp.

2) Preparation of Expression Vector

pHHI plasmid was cleaved with restriction enzymes EcoRI and HindIII and electrophoresed on 1% agarose gel to isolate a DNA segment of 3.1 kbp with tac promoter and the proinsulin fusion protein gene removed.

3) Cloning

The two DNA segments prepared as above were joined together using T4 DNA ligase to produce the plasmid. E. coli JM109 was transformed with the produced vector by the calcium chloride method. The transformed cells resistant to ampicillin were selected. The vector DNA was isolated from each transformant and confirmed that the desired DNA had been properly inserted using an analysis by restriction enzyme cleavage.

### Experiment Example 1

### Expression of Proinsulin Fusion Proteins Using Inventive Plasmids

The expression of the proinsulin fusion proteins using the plasmids according to the present invention was examined as follows.

E. coli was transformed with each of the pK-BKpi type, pPT-B5Kpi, pPL-B5Kpi and pPLD-B5Kpi plasmids prepared in Examples 1, 2 and 4 according to the present invention. Here, as control, E. coli BL21(DE3) was transformed with methionine-lysine-proinsulin expression plasmid, one of insulin expression plasmids used before (Jin et al., 1995). The microorganisms were subjected to the fed batch fermentation as follows.

The cells stored in 20% glycerol at −70° C. were rapidly thawed in purified water at about 30° C., inoculated into 600 ml of LB (Luria-Bertani) medium in a 7 l round flask and cultured under conditions of 37° C. and 250 rpm for 7 hours for seed culture.

The culture fluid was inoculated into 140 l of the initial medium in a 300 l fermentor (B. Braun, Biostat D-300, Germany) and cultured under 200 to 500 rpm, ventilation rate of 1 VVM, temperature of 37° C., pH 6.8 to 7.0, and dissolved oxygen of 30%.

The composition used for the fed batch fermentation is shown in Table 1 below.

TABLE 1

| Component | Initial medium (g/l) | Feed medium (g/l) |
|---|---|---|
| Na₂HPO₄ | 8 | . |
| KH₂PO₄ | 4 | . |
| MgSO₄.7H₂O | 2 | 10 |
| Glucose | 6 | 400 |
| Yeast extract | 4 | 100 |
| Kanamycin (*) | 0.02 | . |
| Trace elements | Trace | Trace |

(*); Ampicillin is used in the medium composition of E. coli JM109/pPT-B5Kpi.

When the glucose level in the initial medium was lowered under 0.1%, glucose was supplied and the level was maintained under 0.01% to keep up the growth of the cells. When the absorption at 600 nm reached about 60, 0.5 mM IPTG was added to induce protein expression. Then, the cells were recovered and the expression of the proinsulin fusion protein was measured (FIG. 4).

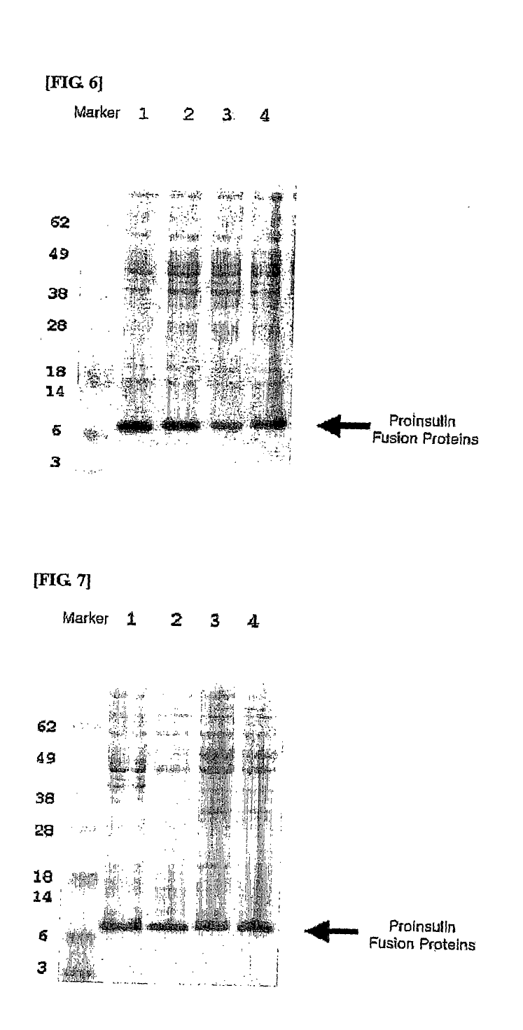

As shown in FIG. 6, the pK-BKpi type plasmids (Line 2: pK-B5Kpi, Line 3: pK-B9Kpi, Line 4: pK-B13Kpi) according to the present invention showed the proinsulin fusion protein expression levels as high as the control which was known to show a high expression level, that is, methionine-lysine-proinsulin (Line 1). Also, as shown in FIG. 7, the plasmids containing P2 or rac promoter (Line 2: pPT-B5Kpi, Line 3: pPL-B5Kpi, Line 4: pPLD-B5Kpi) according to the present invention showed expression levels of the target protein similar to that of pK-B5Kpi plasmid containing T7 promoter (Line 1).

Therefore, it was noted that the plasmids according to the present invention could express the proinsulin fusion protein at a high level.

### Experiment Example 2

### Examination of the By-products Generation in Preparation of Insulin Using Inventive Plasmids

The generation of by-products in the preparation of insulin using the plasmids according to the present invention was examined as follows.

1) Fed Batch Fermentation of E. coli Transformed with Inventive Plasmids

E. coli BL21(DE3) transformed with the pK-B5Kpi plasmid according to the present invention (hereinafter referred to as ‘BL21(DE3)/pK-B5Kpi’) and E. coli JM109 transformed with the pPT-17Kpi plasmid according to the present invention (hereinafter referred to as ‘JM109/pPT-17Kpi’) were subjected to the fed batch fermentation following the method of Experiment Example 1. The E. coli capable of expressing methionine-lysine-proinsulin fusion protein was also prepared and cultured following the method of Experiment Example 1.

The cells obtained from each fermentation were measured for the expression level of the proinsulin fusion proteins and the results are shown in Table 2.

TABLE 2

| | Absorption | | | |
|---|---|---|---|---|
| | upon induction of | Final | Dry cell | Expression |
| | expression | absorption | weight | level |
| E. coli/plasmid | (600 nm) | (600 nm) | (g/l) | (%) |
| BL21(DE3)/pK- | 60 | 106 | 33 | 38 |
| B1Kpi | | | | |
| BL21(DE3)/pK- | 60 | 109 | 33 | 35 |
| B5Kpi | | | | |
| JM109/pPT- | 60 | 106 | 32 | 30 |
| 17Kpi | | | | |

As seen from Table 2, the cells transformed with the plasmids according to the present invention showed high proinsulin fusion protein expression levels.

2) Purification and Sulfonation of Proinsulin Fusion Protein

To the cultured cells, a buffer solution (10% sucrose, 0.1M Tris, 50 mM EDTA, 0.2M sodium chloride, pH 7.9) was added and the cells were lysed under a pressure of about 13,000 to 14,000psi using a homogenizer (Rannie, 14.56VH, Denmark).

The disrupted cells were centrifuged with a continuous centrifuge (Tomo-e, AS-46NF, Japan) at 10,000 rpm. Soluble proteins and a part of cell debris were removed to separate precipitates containing inclusion bodies.

The separated inclusion bodies were washed with a solution containing 2% Triton X-100 and 1M urea and centrifuged at 10,000 rpm to obtain precipitates.

The inclusion bodies were dissolved in a solution at pH 9.0 containing 8M urea, 20 mM Tris and 1 mM EDTA in a volume of 15 times of the wet weight of the purified inclusion bodies, and sodium sulfite and sodium tetrationate were added to final concentration of 0.2 to 0.4M and 20 to 100 mM, respectively. Here, the added amounts of sodium sulfite and sodium tetrationate are preferably 0.2M and 20 mM, respectively.

The resulting solution was stirred for 12 hours at 4° C. for sulfonation of cystein residues in the proinsulin fusion protein and centrifuged at 12,000 rpm to remove insoluble precipitates.

3) Direct Refolding of Sulfonated Proinsulin Fusion Protein

The supernatant from the centrifugation containing the sulfonated proinsulin fusion protein was diluted in purified water to a final protein level of 1 mg/ml, deaerated with nitrogen gas, and sealed.

Separately, to a glycine buffer solution (0.6M urea, 50 mM glycine, pH 10.6) in an equal volume to the above, β-mercaptoethanol was added to 1.5 equivalent of insulin cysteine residues, then the solution was deaerated with nitrogen gas and sealed.

The two solutions were rapidly mixed in a mixing rate (v/v) of 1:1 and incubated at 10° C. for 18 hours to perform the refolding.

In order to comparatively examine the direct refolding yield of the method for preparing insulin according to the present invention and the amount of the refolded proinsulin fusion protein, the sulfonated proinsulin fusion protein prepared from the above was purified by chromatography and subjected to refolding, which was then used as control. The results are shown in Table 3.

TABLE 3

| | | Refolded proinsulin |
|---|---|---|
| Sulfonated proinsulin | Refolding yield | fusion protein |
| fusion protein | (%) | (mg/l) |
| Refolding after chromatography | 65 | 360 |
| Direct refolding | 62 | 383 |

As seen from Table 3, it was noted that the yield of the direct refolding according to the present invention was about 62% similar to the yield of the refolding after purification by chromatography which had been performed. Also, the production of the refolded proinsulin fusion protein was much higher than the conventional technology.

Therefore, it was noted that it is possible to effectively perform the refolding of the proinsulin fusion protein by the method for preparing insulin using the plasmids according to the present invention.

4) Conversion into Active Insulin by Enzymatic Cleavage

To the refolded proinsulin fusion protein solution as described above, 20 mM Tris was added, then trypsin 0.45 u and carboxypeptidase B 0.2 u per protein 1 mg were added at pH 7.5 and the solution was incubated at 15° C. for 16 hours.

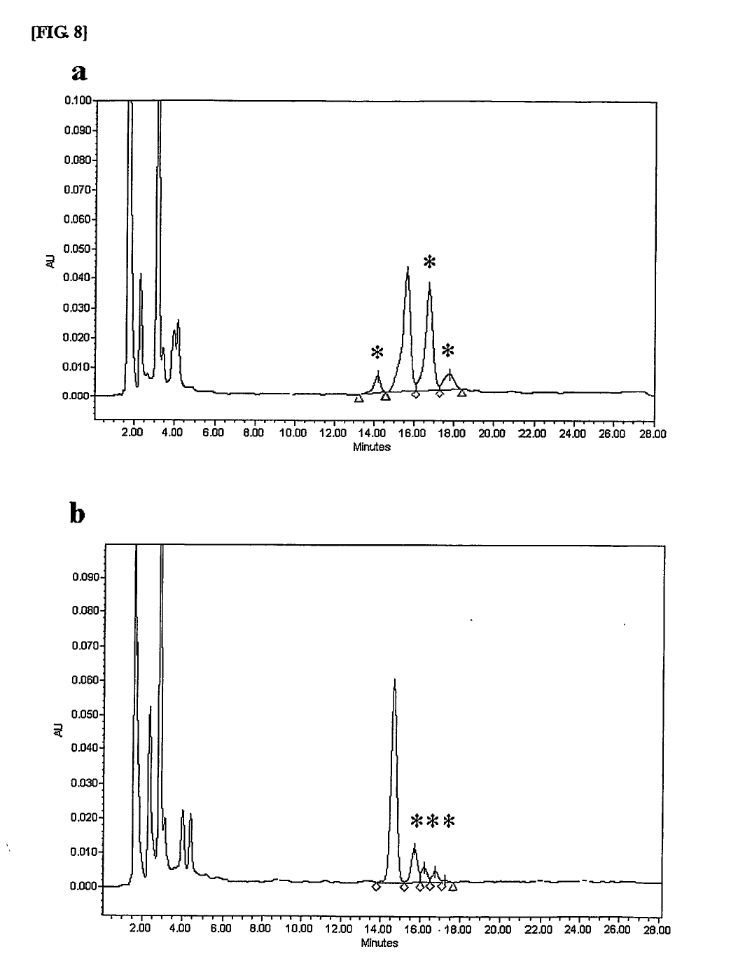

In order to measure the amount of insulin by-products generated in the above process, reverse phase high pressure liquid chromatography was performed and the results are shown in FIG. 8 (the insulin by-products were marked with *) As shown in 8a, in the expression of methionine-lysine-proinsulin, a large amount of insulin by-products was generated after the enzymatic cleavage by trypsin and carboxypeptidase B.

On the other hand, as shown in FIG. 8b, in the expression of the pK-B5Kpi splasmid according to the present invention, the level of insulin by-products was low while the insulin production was very high.

The numerical results are shown in Table 4 below.

TABLE 4

| E. coli/plasmid | Insulin (%) | Insulin by-products (%) |
|---|---|---|
| BL21(DE3)/pK-B1Kpi | 48 | 39 |
| BL21(DE3)/pK-B5Kpi | 75 | 11 |
| JM109/pPT-17Kpi | 71 | 15 |

Therefore, since the plasmids according to the present invention generate a small amount of the insulin by-products, they don't need an additional purification due to the mass-generation of the by-products, thereby effectively producing insulin.

### Experiment Example 3

### Preparation of Insulin without Sulfonation

Insulin was prepared using the pPT-B5Kpi plasmid according to the present invention without sulfonation process.

1) Fed Batch Fermentation of E. coli Transformed with Inventive Plasmid

E. coli JM109 transformed with the pPT-B5Kpi, prepared in Example 2, was subjected to the fed batch fermentation. The fermentation was performed following the method of Experiment Example 1.

2) Purification of Proinsulin Fusion Protein

To the cultured cells, a buffer solution (10% sucrose, 0.1M Tris, 50 mM EDTA, 0.2M sodium chloride, pH 7.9) was added and the cells were disrupted using a homogenizer. In order to minimize the loss of precipitates containing inclusion bodies and increase the yield during centrifugation, the disrupted cells were set to a low temperature (10° C.) and an acid condition (pH 5.0).

By centrifugation using a continuous centrifuge, precipitates containing the inclusion bodies were recovered. The inclusion bodies were suspended with a solution at pH 7.0 containing 20 mM Tris and 1 mM EDTA to control the washing condition.

The product was washed with 1% Triton X-100 for 2 hours to remove fat and membrane proteins and washed with 2M urea for 3 hours to remove proteins attached to the inclusion bodies.

When the inclusion bodies were washed by the set-forth improved method and recovered by centrifugation, the amount of the recovered inclusion bodies were increased and the purity was also improved.

3) Refolding of Proinsulin Fusion Protein without Sulfonation

The purified inclusion bodies were completely dissolved in a solution at pH 9.0 containing 8M urea in a volume of 20 times of the wet weight of the inclusion bodies and diluted 20 times with water. Then, β-mercaptoethanol was added to the resulting solution to a concentration of 0.25 mM, and the solution was set to pH 10.6 and stirred at 4° C. for 12 hours to perform the refolding.

4) Conversion into Active Insulin by Enzymatic Cleavage

The refolded proinsulin fusion protein solution as described above was treated with trypsin and carboxypeptidase B following the method of Experiment Example 2-4).

In comparison with the refolding after sulfonation as the method of Experiment Example 2-3), the refolding as described above showed comparable results in terms of insulin conversion yield and purity, and thus it was confirmed that the insulin production can be further simplified.

5) Conclusion

Therefore, by the plasmid according to the present invention and the method for preparing insulin using the same, it is possible to produce insulin at a high yield in a much simpler way, as compared to the prior art, while minimizing the generation of by-products.

### Experiment Example 4

### Preparation of Insulin by Immobilized Enzymes

1) Immobilization of Enzymes

10 g of Amberlite XAD-7 was washed with methanol, 25% hydroperoxide and 5% nitric acid, treated with 50 ml of ethylenediamine for 4 hours, washed with water and dried.

To the resulting resin, 230 ml of 3% glutaldehyde dissolved in phosphate buffer solution (pH 7.5) was added and reacted at 20° C. for 1 hours. The resin was washed with 0.02M phosphate buffer solution (pH 7.5).

Also, to the resulting resin, 400 ml of 0.1M phosphate buffer solution (pH 7.5) with trypsin 200 u/ml or carboxypeptidase B 100 u/ml was added and stirred at 20° C. for 2 hours. The resin was washed with 0.02M phosphate buffer solution.

Then, to the resulting resin, 400 ml of phosphate buffer solution containing sodium borohydride 0.06 g was added, stirred at 20° C. for 1 hour and washed with the buffer solution to prepare immobilized trypsin and immobilized carboxypeptidase B.

2) Conversion into Active Insulin by Immobilized Enzymes

For the enzymatic reaction, the refolded proinsulin fusion protein was dissolved in 20 mM Tris solution (pH 7.5) to a concentration of 0.5 mg/ml, then the immobilized trypsin 2000 u and the immobilized carboxypeptidase B 1000 u per protein 1 mg were added to the solution and reacted at 15° C.

3) Conclusion

According to this example, it was noted that the insulin conversion yield and the purity were comparable to the results from the Experimental Examples 2 and 3.

Therefore, it would be possible to efficiently produce insulin by using the immobilized enzymes according to the present invention.

By the plasmids according to the present invention and the method for preparing insulin using the same, it is possible to convert the proinsulin fusion protein into human insulin in a single process.

Also, by the plasmids according to the present invention and the method for preparing insulin using the same, it is possible to minimize the generation of by-products, thereby producing insulin at a high yield.

Thus, the plasmids according to the present invention and the method for preparing insulin using the same can be usefully applied to the industrial mass-production of human insulin.

1. A plasmid comprising a sequence encoding a compound of the following formula (I):
R—B—X-A (I)
in which,
(i) R is a leader peptide represented by the following formula (II):

| | |
|---|---|
| Met-Thr-Met-Ile-Thr-Y (II) | (SEQ ID NO: 36) |

in which Y is one selected from lysine, arginine, a peptide containing lysine as an amino acid at its C-terminal, or a peptide containing arginine as an amino acid at its C-terminal;
(ii) B is human insulin B-chain or analogue thereof;
(iii) X is a peptide connecting B with A; and
(iv) A is human insulin A-chain or analogue thereof.

2. The plasmid according to claim 1, in which the R of the formula (I) is selected from the following peptide sequences of SEQ ID NOs. 1, 2, 3, 4, 5, 6, 7 or 8:

| | |
|---|---|
| Met-Thr-Met-Ile-Thr-Lys: | SEQ ID NO. 1 |
| Met-Thr-Met-Ile-Thr-Asp-Ser-Leu-Ala- | SEQ ID NO. 2 |
| Lys: | |
| Met-Thr-Met-Ile-Thr-Asp-Ser-Leu-Ala- | SEQ ID NO. 3 |
| Val-Val-Leu-Gln-Lys: | |
| Met-Thr-Met-Ile-Thr-Asp-Ser-Leu-Ala- | SEQ ID NO. 4 |
| Val-Val-Leu-Gln-Gly-Ser-Leu-Gln-Lys: | |
| Met-Thr-Met-Ile-Thr-Arg: | SEQ ID NO. 5 |
| Met-Thr-Met-Ile-Thr-Asp-Ser-Leu-Ala- | SEQ ID NO. 6 |
| Arg: | |
| Met-Thr-Met-Ile-Thr-Asp-Ser-Leu-Ala- | SEQ ID NO. 7 |
| Val-Val-Leu-Gln-Arg: | |
| Met-Thr-Met-Ile-Thr-Asp-Ser-Leu-Ala- | SEQ ID NO. 8 |
| Val-Val-Leu-Gln-Gly-Ser-Leu-Gln-Arg: | |

3. The plasmid according to claim 1, in which the peptide sequences of SEQ ID NOs. 1 to 8 are encoded by the following sequences of SEQ ID NOs. 9 to 16:

| | |
|---|---|
| ATG ACC ATG ATT ACG AAG: | SEQ ID NO. 9 |
| ATG ACC ATG ATT ACG GAT TCA CTG GCC | SEQ ID NO. 10 |
| AAG: | |
| ATG ACC ATG ATT ACG GAT TCA CTG GCC | SEQ ID NO. 11 |
| GTC GTT TTA CAA AAG: | |
| ATG ACC ATG ATT ACG GAT TCA CTG GCA | SEQ ID NO. 12 |
| GTC GTT TTA CAA GGT TCT CTG CAG AAG: | |
| ATG ACC ATG ATT ACG CGT: | SEQ ID NO. 13 |
| ATG ACC ATG ATT ACG GAT TCA CTG GCC | SEQ ID NO. 14 |
| CGT: | |
| ATG ACC ATG ATT ACG GAT TCA CTG GCC | SEQ ID NO. 15 |
| GTC GTT TTA CAA CGT: | |
| ATG ACC ATG ATT ACG GAT TCA CTG GCA | SEQ ID NO. 16 |
| GTC GTT TTA CAA GGT TCT CTG CAG CGT: | |

4. The plasmid according to claim 2 or 3, in which the B, X and A of the formula (I) are human insulin B-chain, C-chain and A-chain, respectively.

5. The plasmid according to claim 2 or 3, in which the B of the formula (I) is a peptide having the residues Nos. 28 and 29 of human insulin B-chain exchanged to each other, and X and A are human insulin C-chain and A-chain, respectively.

6. The plasmid according to claim 4, which the plasmid has the structure of FIG. 1c.

7. The plasmid according to claim 6, in which the plasmid is selected from pK-B5Kpi, pK-B9Kpi, pK-B13Kpi, pK-B5Rpi, pK-B9Rpi and pK-B13Rpi.

8. The plasmid according to claim 7, in which the plasmid is pK-B5Kpi plasmid deposited under accession No. KCTC 10363BP.

9. The plasmid according to claim 4, in which the plasmid has the structure of FIG. 3c.

10. The plasmid according to claim 9, in which the plasmid is selected from pPT-B5Kpi, pPT-B9Kpi, pPT-B13Kpi, pPT-B5Rpi, pPT-B9Rpi, pPT-B13Rpi, pPT-17Kpi and pPT-17Rpi.

11. The plasmid according to claim 4, in which the plasmid has the structure of FIG. 4c.

12. The plasmid according to claim 11, in which the plasmid selected from pPL-B5Kpi, pPL-B9Kpi, pPL-B13Kpi, pPLD-B5Kpi, pPLD-B9Kpi, pPLD-B13Kpi, pPL-B5Rpi, pPL-B9Rpi, pPL-B13Rpi, pPLD-B5Rpi, pPLD-B9Rpi and pPLD-B13Rpi.

13. The plasmid according to claim 5, in which the plasmid has the structure of FIG. 3c.

14. The plasmid according to claim 13, in which the plasmid is selected from pPT-B5KpiKP, pPT-B9KpiKP, pPT-B13KpiKP, pPT-B5RpiKP, pPT-B9RpiKP and pPT-B13RpiKP.

15. A microorganism transformed with the plasmid according to any one of claims 6 to 14.

16. A method for preparing human insulin or a analogue thereof comprising:
(a) a step to induce the expression of a compound of the following formula (I) by fermenting the microorganism of claim 15:
R—B—X-A (I)
in which
(i) R is a leader peptide represented by the following formula (II):

| | |
|---|---|
| Met-Thr-Met-Ile-Thr-Y (II) | (SEQ ID NO: 36) |

in which, Y is one selected from lysine, arginine, a peptide containing lysine as an amino acid at its C-terminal, or a peptide containing arginine as an amino acid at its C-terminal;
(ii) B is human insulin B-chain or analogue thereof;
(iii) X is a peptide connecting B with A; and
(iv) A is human insulin A-chain or analogue thereof;
(b) a step of cell disruption and dissolution;
(c) a step of refolding;
(d) a step of co-cleavage of R and X by an enzymatic reaction; and
(e) a step of purification of active insulin by chromatography.

17. The method according to claim 16, in which the (d) step is performed at pH 7 to 8, a reaction temperature of 4° to 28°, trypsin level per protein 1 mg of 0.1 u to 0.5 u, carboxypeptidase B level per protein 1 mg of 0.1 u to 0.3 u, a reaction time of 12 to 24 hours.

18. The method according to claim 16, in which the (d) step is performed using both immobilized trypsin and immobilized carboxypeptidase B.


SEQUENCE LISTING (35 sequences)

SEQ ID NO: 1 - protein, 6 aa, Artificial Sequence
  amino acid sequence for leader peptide 1
MTMITK

SEQ ID NO: 2 - protein, 10 aa, Artificial Sequence
  amino acid sequence for leader peptide 2
MTMITDSLAK

SEQ ID NO: 3 - protein, 14 aa, Artificial Sequence
  amino acid sequence for leader peptide 3
MTMITDSLAVVLQK

SEQ ID NO: 4 - protein, 18 aa, Artificial Sequence
  amino acid sequence for leader peptide 4
MTMITDSLAVVLQGSLQK

SEQ ID NO: 5 - protein, 6 aa, Artificial Sequence
  amino acid sequence for leader peptide 5
MTMITR

SEQ ID NO: 6 - protein, 10 aa, Artificial Sequence
  amino acid sequence for leader peptide 6
MTMITDSLAR

SEQ ID NO: 7 - protein, 14 aa, Artificial Sequence
  amino acid sequence for leader peptide 7
MTMITDSLAVVLQR

SEQ ID NO: 8 - protein, 18 aa, Artificial Sequence
  amino acid sequence for leader peptide 8
MTMITDSLAVVLQGSLQR

SEQ ID NO: 9 - DNA, 18 bp, Artificial Sequence
  nucleotide sequence encoding leader peptide 1
atgaccatgattacgaag

SEQ ID NO: 10 - DNA, 30 bp, Artificial Sequence
  nucleotide sequence encoding leader peptide 2
atgaccatgattacggattcactggccaag

SEQ ID NO: 11 - DNA, 42 bp, Artificial Sequence
  nucleotide sequence encoding leader peptide 3
atgaccatgattacggattcactggccgtcgttttacaaaag

SEQ ID NO: 12 - DNA, 54 bp, Artificial Sequence
  nucleotide sequence encoding leader peptide 4
atgaccatgattacggattcactggcagtcgttttacaaggttctctgcagaag

SEQ ID NO: 13 - DNA, 18 bp, Artificial Sequence
  nucleotide sequence encoding leader peptide 5
atgaccatgattacgcgt

SEQ ID NO: 14 - DNA, 30 bp, Artificial Sequence
  nucleotide sequence encoding leader peptide 6
atgaccatgattacggattcactggcccgt

SEQ ID NO: 15 - DNA, 42 bp, Artificial Sequence
  nucleotide sequence encoding leader peptide 7
atgaccatgattacggattcactggccgtcgttttacaacgt

SEQ ID NO: 16 - DNA, 54 bp, Artificial Sequence
  nucleotide sequence encoding leader peptide 8
atgaccatgattacggattcactggcagtcgttttacaaggttctctgcagcgt

SEQ ID NO: 17 - DNA, 46 bp, Artificial Sequence
  forward primer for amplifying nucleotide sequence encoding leader peptide 1
caccagcatatgaccatgattacgaagtttgtgaaccaacacctgt

SEQ ID NO: 18 - DNA, 60 bp, Artificial Sequence
  forward primer for amplifying nucleotide sequence encoding leader peptide 2
caccagcatatgaccatgattacggattcactggccaagtttgtgaaccaacacctgtgc

SEQ ID NO: 19 - DNA, 72 bp, Artificial Sequence
  forward primer for amplifying nucleotide sequence encoding leader peptide 3
caccagcatatgaccatgattacggattcactggccgtcgttttacaaaagtttgtgaaccaacacctgtgc

SEQ ID NO: 20 - DNA, 46 bp, Artificial Sequence
  forward primer for amplifying nucleotide sequence encoding leader peptide 5
caccagcatatgaccatgattacgcgttttgtgaaccaacacctgt

SEQ ID NO: 21 - DNA, 60 bp, Artificial Sequence
  forward primer for amplifying nucleotide sequence encoding leader peptide 6
caccagcatatgaccatgattacggattcactggcccgttttgtgaaccaacacctgtgc

SEQ ID NO: 22 - DNA, 72 bp, Artificial Sequence
  forward primer for amplifying nucleotide sequence encoding leader peptide 7
caccagcatatgaccatgattacggattcactggccgtcgttttacaacgttttgtgaaccaacacctgtgc

SEQ ID NO: 23 - DNA, 24 bp, Artificial Sequence
  reverse primer for amplifying Xho I restriction site of pHHI plasmid
gcatgcctcgaggtcgactctaga

SEQ ID NO: 24 - DNA, 91 bp, Artificial Sequence
  forward primer for amplifying EcoRI restriction site and upstream region of P2 promoter
catgttgaattctgcgccaccactgacacggacaacggcaaacacgccgccgggtcagcggggttctcctgagaactccggcagagaaagc

SEQ ID NO: 25 - DNA, 96 bp, Artificial Sequence
  reverse primer for amplifying -35 and -10 region of P2 promoter,and lac operator
tgtttcctgtgtgaaattgttatccgctcacaattccataatacgccttcccgctacagagtcaagcatttatttttgctttctctgccggagttc

SEQ ID NO: 26 - DNA, 96 bp, Artificial Sequence
  reverse primer for amplifying T7 ribosome binding site, and NdeI,KpnI, XhoI, SalI and HindIII restriction site
acagccaagcttgtcgactcgaggtaccgacatatgtatatctccttcttaaagttaaacaaaattatttctagaagctgtttcctgtgtgaaatt

SEQ ID NO: 27 - DNA, 83 bp, Artificial Sequence
  forward primer for amplifying nucleotide sequence encoding leader peptide 4
gaaacacatatgaccatgattacggattcactggcagtcgttttacaaggttctctgcagaagtttgtgaaccaacacctgtg

SEQ ID NO: 28 - DNA, 83 bp, Artificial Sequence
  forward primer for amplifying nucleotide sequence encoding leader peptide 8
gaaacacatatgaccatgattacggattcactggcagtcgttttacaaggttctctgcagcgttttgtgaaccaacacctgtg

SEQ ID NO: 29 - DNA, 20 bp, Artificial Sequence
  forward primer complementary to +60 upstream region from EcoRI restriction site of pPT-B5Kpi plasmid
agtaaggcaaccccgccagc

SEQ ID NO: 30 - DNA, 49 bp, Artificial Sequence
  reverse primer for amplifying -35 region of P2 promoter, -10 region of lac promoter, and part of lac operator to be used in the construction of pPL vector
ttatccgctcacaattccacacaacatacgagccttcccgctacagagt

SEQ ID NO: 31 - DNA, 45 bp, Artificial Sequence
  reverse primer for amplifying -35 region of P2 promoter, -100 region of lac promoter, and part of lac operator to be used in the construction of pPLD vector
ttatccgctcacaattccaacatacgagccttcccgctacagagt

SEQ ID NO: 32 - DNA, 78 bp, Artificial Sequence
  reverse primer for amplifying T7 ribosome binding site and NdeI restriction site
taccgacatatgtatatctccttcttaaagttaaacaaaattatttctagagggaaattgttatccgctcacaattcc

SEQ ID NO: 33 - DNA, 33 bp, Artificial Sequence
  primer for interchanging 28th with 29th in amino acid sequence of insulin B chain
ctcccggcgggtgggctttgtgtagaagaagcc

SEQ ID NO: 34 - DNA, 33 bp, Artificial Sequence
  primer complementary to the sequence of primer 25
ggcttcttctacacaaagcccacccgccgggag

SEQ ID NO: 35 - DNA, 20 bp, Artificial Sequence
  reverse primer complementary to -80 downstream region from HindIII restriction site
ctgccgccaggcaaattctg